(19) United States
(12) Patent Application Publication
(10) Pub. No.: US 2010/0010328 A1
(43) Pub. Date: Jan. 14, 2010

(54) PROBES AND SENSORS FOR ASCERTAINING BLOOD CHARACTERISTICS AND METHODS AND DEVICES FOR USE THEREWITH

(76) Inventors: Harry D. Nguyen, Westminster, CA (US); Jim F. Martin, Woodside, CA (US); Margaret R. Webber, Los Altos, CA (US); Paul Douglas Corl, Palo Alto, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/172,181

(22) Filed: Jul. 11, 2008

(51) Int. Cl. A61B 5/1468

(52) U.S. Cl. 600/354; 600/364; 600/361

(57) ABSTRACT

A probe for use in a patient having a vessel carrying blood to ascertain characteristics of the blood having a cannula adapted to be inserted into the vessel of the patient. The cannula has a length so that when the distal extremity is in the vessel of the patient the proximal extremity is accessible outside of the patient. A gas sensor assembly is carried within the distal extremity of the cannula for determining gas characteristics of the blood in the vessel. A pressure sensor is carried within the distal extremity of the cannula for determining the pressure of the blood in the vessel.

### BACKGROUND OF THE INVENTION

1. Technical Field

This invention relates to probes for measuring physiological parameters in a mammalian body and, in particular, to probes for ascertaining characteristics of blood in a mammalian body.

2. Description of the Related Art

Determination of cardiac output, arterial blood gases, blood pressure and other hemodynamic or cardiovascular parameters is critically important in the treatment and care of patients, particularly those undergoing surgery or other complicated medical procedures and those under intensive care. Typically, cardiac output measurements have been made using pulmonary artery thermodilution catheters, which can have inaccuracies of 20% or greater. It has been found that the use of such thermodilution catheters increases hospital costs while exposing the patient to potential infectious, arrhythmogenic, mechanical, and therapeutic misadventure. Blood gas measurements have also heretofore been made. Commonly used blood gas measurement techniques require a blood sample to be removed from the patient and transported to a lab analyzer for analysis. The caregiver must then wait for the results to be reported by the lab, a delay of 20 minutes being typical and longer waits not unusual.

More recent advances in the art have provided for “point-of-care” blood testing systems wherein testing of blood samples is performed at a patient's bedside or in the area where the patient is located. Such systems include portable and handheld units and modular units which fit into a bedside monitor. While most point-of-care systems require the removal of blood from the patient for bedside analysis, a few do not. In such systems, intermittent blood gas measurements are made by drawing a sufficiently large blood sample into an arterial line to ensure an undiluted sample at a sensor located in the line. After analysis, the blood is returned to the patient, the line is flushed, and results appear on the bedside monitor.

A non-invasive technology, pulse oximetry, is available for estimating the percentage of hemoglobin in arterial blood that is saturated with oxygen. Although pulse oximeters are capable of estimating arterial blood oxygen content, they are not capable of measuring carbon dioxide, pH, or venous oxygen content. Furthermore, pulse oximetry is commonly performed at the fingertip and can be skewed by peripheral vasoconstriction or even nail polish.

Blood pressure can be measured non-invasively using a blood pressure manometer connected to an inflatable cuff. This is the most common method outside of the intensive care environment. In critical care settings, at least 60% of patients have arterial lines. An arterial line consists of a plastic cannula inserted into a peripheral artery (commonly the radial or the femoral). The cannula is kept open and patent because it is connected to a pressurized bag of heparinized fluid such as normal saline. An external gauge also connects to the arterial cannula to reflect the column of fluid pressure in the artery. This system consists of an arterial line connected by saline filled non-compressible tubing to a pressure transducer. This converts the pressure waveform into an electrical signal which is displayed on the bedside monitor. The pressurized saline for flushing is provided by a pressure bag.

There are several potential sources of error in this system. First, any one of the components in the system can fail. Second, the transducer position is critical because the pressure displayed is pressure relative to position of transducer. Thus, in order to accurately reflect blood pressure, the transducer should be at the level of the heart. Over-reading will occur if transducer too low and under-reading if transducer too high. Third, the transducer must be zeroed to the atmospheric pressure at the time of measurement, otherwise, the blood pressure will be incorrectly measured.

Fourth, it is critical to have appropriate damping in the system. Inadequate damping will result in excessive resonance in the system, which causes an overestimate of systolic pressure and an underestimate of diastolic pressure. The opposite occurs with over-damping. In both cases the mean arterial pressure is the most accurate. An under-damped trace is often characterized by a high initial spike in the waveform.

Unfortunately, none of the available systems or methods for blood gas analysis provides for accurate, direct and continuous in vivo measurements of arterial and venous oxygen partial pressures, carbon-dioxide partial pressure, pH, cardiac output, and blood pressure while presenting minimal risk to the patient.

### SUMMARY OF THE INVENTION

A probe for use in a patient having a vessel carrying blood to ascertain characteristics of the blood having a cannula adapted to be inserted into the vessel of the patient is provided. The cannula has a length so that when the distal extremity is in the vessel of the patient the proximal extremity is accessible outside of the patient. A gas sensor assembly is carried within the distal extremity of the cannula for determining gas characteristics of the blood in the vessel. A pressure sensor is carried within the distal extremity of the cannula for determining the pressure of the blood in the vessel.

### BRIEF DESCRIPTION OF THE DRAWINGS

For a better understanding of the nature and details of the invention, reference should be made to the following drawings, which in some instances are schematic in detail and wherein like reference numerals have been used throughout.

FIG. 1 is an isometric view of a probe for ascertaining blood characteristics of the present invention coupled to a display module.

FIG. 2 is a cutaway and partially sectioned view of the connector portion of one embodiment of a probe.

FIGS. 2A, 2B and 2C are a section view and two plan views, respectively, of an alternative and preferred version of the connector portion of one embodiment of a probe.

FIG. 3 is an enlarged cross-sectional view of the pH sensor section of one embodiment of a probe.

FIG. 4 is an enlarged cross-sectional view of the carbon dioxide sensor section of one embodiment of a probe.

FIG. 5 is an enlarged cross-sectional view of the oxygen sensor section of one embodiment of a probe.

FIG. 5A is an enlarged cross-sectional view of an alternative and preferred version of the oxygen sensor section of one embodiment of a probe.

FIG. 6A is an enlarged cross-sectional view of one embodiment of the blood pressure sensor section of a probe.

FIG. 6B is a cross-sectional view of the blood pressure sensor section, orthogonal to FIG. 6A.

FIG. 6C is a cross-sectional view of the blood pressure sensor section, orthogonal to FIGS. 6A and 6B.

FIG. 7 is a side elevational view of another embodiment of a probe for ascertaining blood characteristics.

FIG. 8 is a plan view of the top of the probe of FIG. 7.

FIG. 9 is a plan view of the bottom of the first layer of the probe of FIG. 7.

FIG. 10 is a plan view of the second layer of the probe of FIG. 5.

FIG. 11 is a plan view of the top of the third layer of the probe of FIG. 5.

FIG. 12 is a bottom plan view of the probe of FIG. 7.

FIG. 13 is an isometric view of another embodiment of a probe for ascertaining blood characteristics.

FIG. 14 is a plan view, partially cut away, of a kit of the present invention.

### DETAILED DESCRIPTION OF THE PREFERRED EMBODIMENT

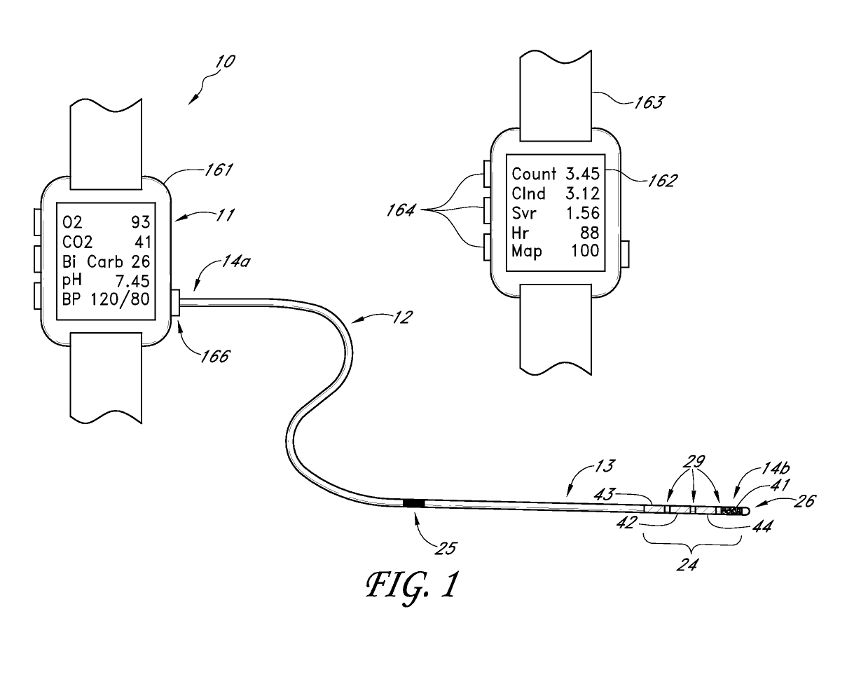

An apparatus 10 according to the present invention for making intravascular measurement of physiological parameters or characteristics generally includes, as shown in FIG. 1, a display module 11 and one or more probes 12. As described in more detail herein, the display module 11 and probe 12 are particularly adapted for accurate and continuous in vivo measurement and display of intravascular parameters such as partial pressure of oxygen (PO2), partial pressure of carbon dioxide (PCO2), pH and blood pressure. In addition, cardiac output (CO) can be calculated by combining two measurements of PO2 obtained from a pair of probes, one disposed in an artery and the other in a vein. Alternatively, or in addition to the aforementioned sensors, the probe 12 may include sensors for other useful blood parameters such as potassium, sodium, calcium, bilirubin, hemoglobin/hematocrit, glucose and pressure. Additional features of some embodiments of the display module 11 and probe 12 are detailed hereinafter and in copending U.S. patent application Ser. No. 10/658,926 filed Sep. 9, 2003, and U.S. Pat. No. 6,616,614, the entire content of each of which is incorporated herein by this reference.

Probe 12, as shown in FIG. 1, comprises a flexible elongate probe body or cannula 13. The cannula or sleeve 13 is preferably formed of a suitable insulating material such as a plastic, which provides strength and flexibility to the cannula and thus serves as a structural element of the probe 12. A preferred plastic material for the cannula or sleeve 13 is a polymer and more preferably polymethylpentene. Among commonly-used polymers suitable for extrusion as thin-walled tubing, polymethylpentene has among the highest oxygen and carbon dioxide permeability coefficients available. In addition, it has great stiffness. Cannula or sleeve 13 has a proximal extremity or end portion 14a and a distal extremity or end portion 14b, and has a substantially uniform diameter over its entire length, and has a wall thickness ranging from 0.001 to 0.003 inch and preferably approximately 0.0015 inch. The cannula is of sufficient length so that when the distal extremity 14b is in a vessel of the mammalian body for use the proximal extremity 14a is accessible outside of the mammalian body. Probe 12 includes a sensor section 24, a marker band 25 and a blunt tip 26 at the distal end portion or extremity 14b of the probe.

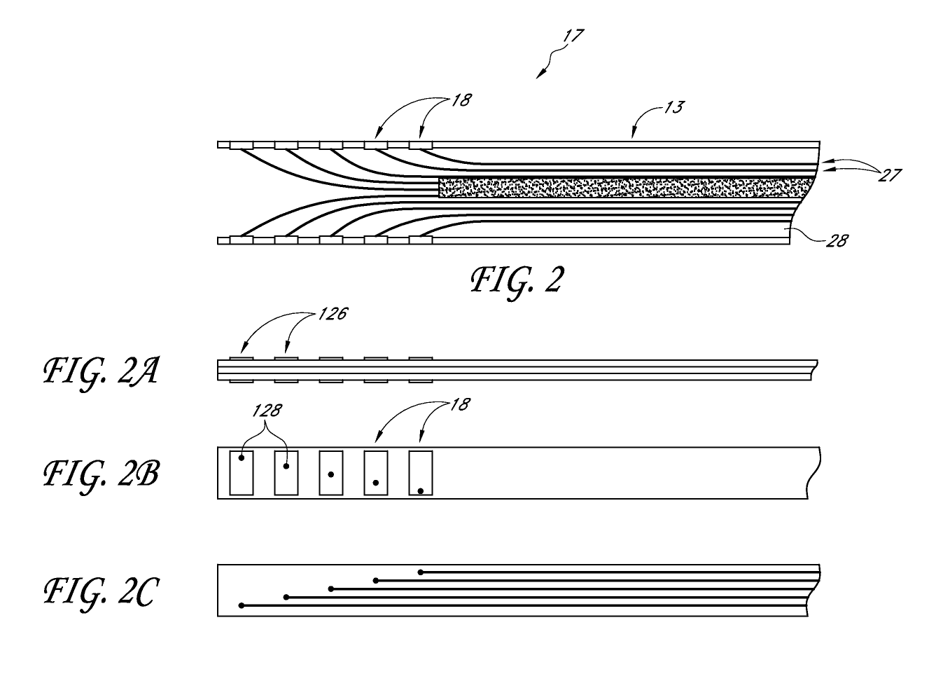

Probe 12 removeably connects to and communicates with display module 11 by way of a suitable probe connector 17, shown in FIG. 2, located at the proximal end 14a of the probe and having a plurality of electrical contacts 18 that are annular or cylindrical in conformation. Additionally, the electrical contacts may be distributed on one or both sides of a flat connector, such as a kind of flexible printed circuit board. Such electrical contacts 18 provide for a low-profile electrical connector 17. The electrical contacts 18 may consist of gold or other suitable bands or pads. A plurality of electrical conductors or conductor means 27 pass through the length of the cannula 13, through a bore or lumen 28, provided in the tubular cannula, and attach to the plurality of contacts 18 of the connector 17 for providing electrical outputs to the proximal extremity 14a of cannula 13. Conductors 27 can each be formed from any suitable conductive material such as copper, platinum or silver, which is covered by an insulating material along its entire length between its exposed ends, and are each of uniform diameter or thickness along its length. Contacts 18 are soldered or welded or otherwise coupled conductively to the electrical conductors 27, which are electrically coupled to the one or more sensors in the sensor section 24 of the probe so as to carry the electrical signals from such multiple sensors and thus permit electrical access to the probe from outside the patient's body. Alternatively, the electrical conductors 27 are formed of specific conductive materials such as platinum or silver, the distal ends of which are formed into the various sensor elements.

One embodiment of probe connector 17 is shown in FIGS. 2A, 2B and 2C. FIG. 2A shows a cross-section view of three layers; each layer is formed of a suitable insulating sheet, such as that used for flexible printed circuits. The top and bottom layers, shown in FIGS. 2B and 2C, respectively, are each plated with suitable conductive material in the form of traces and pads. The traces are connected at their distal ends to electrical conductors 27 by soldering or other conductive means. The traces are connected at their proximal ends to pads on the reverse side of the layer by means of plated vias through the layer, in the conventional means used for flexible printed circuits. The three layers are bonded together as shown in FIG. 2A, with the conventional means used for flexible printed circuits.

Referring back to FIG. 1, a gas permeable window 29 preferably covers at least the oxygen and carbon dioxide portions of the sensor section 24 of the probe 12. In this regard, all or a portion of the body or cannula 13 can also serve as a gas permeable membrane or window 29. The polymer material of the cannula or sleeve 13 permits the passage of oxygen and carbon dioxide gases while blocking the passage of liquid water and the ions dissolved therein when serving as the gas permeable membrane. The cannula or sleeve 13 defines the outer surface of a major portion of the probe 12, and the substantial majority of the cannula or sleeve 13 can be filled with a flexible polymer 33 such as ultraviolet-cured adhesive (referred to also as adhesive encapsulant) to provide robustness to the probe body 13, to anchor the electrical conductors 27 and sensor electrode assemblies inside the sensor section 24, and to seal the ends of any chambers provided in the probe 12 in the vicinity of such sensor electrode assemblies. Alternatively, multiple types of adhesive and other fillers may be utilized to improve either the performance or the ease of assembly of the probe 12. For example, cyanoacrylate can be used for small-scale bonding and small gap filling, and an ultraviolet-cured adhesive can be used for large gap filling and forming chamber walls.

All of the probe elements are dimensioned to fit substantially within the diameter of the probe body 13 such that the entire probe 12, including the low-profile connector 17, may be passed through the inner bore of a suitable introducer, such as a hypodermic needle (not shown), of a size suitable for accessing a blood vessel in the hand, wrist, or forearm. In some embodiments, the probe body 13 has an outer diameter in the range of 0.015 to 0.030 inch. In some embodiments, he probe body 13 has an outer diameter of approximately 0.020 inch. Depending on the diameter of the probe body 13, a suitable hypodermic needle for this purpose may be preferably 20 gauge having an inner diameter of at least 0.023 inch, suitable for use with a probe body having a nominal diameter of 0.020 inch. In some embodiments, the probe 12 can have a suitable length such as 25 centimeters, permitting the sensor section 24 to be inserted into a blood vessel in the hand, wrist, or forearm, while the low-profile connector 17 at the proximal end or extremity of probe 12 is connected to the display module 11, which can be strapped to the wrist of the patient. Marker band 25 is a guide for the insertion of the probe, and is placed preferably 50 mm from the distal end of extremity 14b of the probe 12. When the probe is completely inserted, marker band 25 should be visible just outside the point of entry of the probe into the skin.

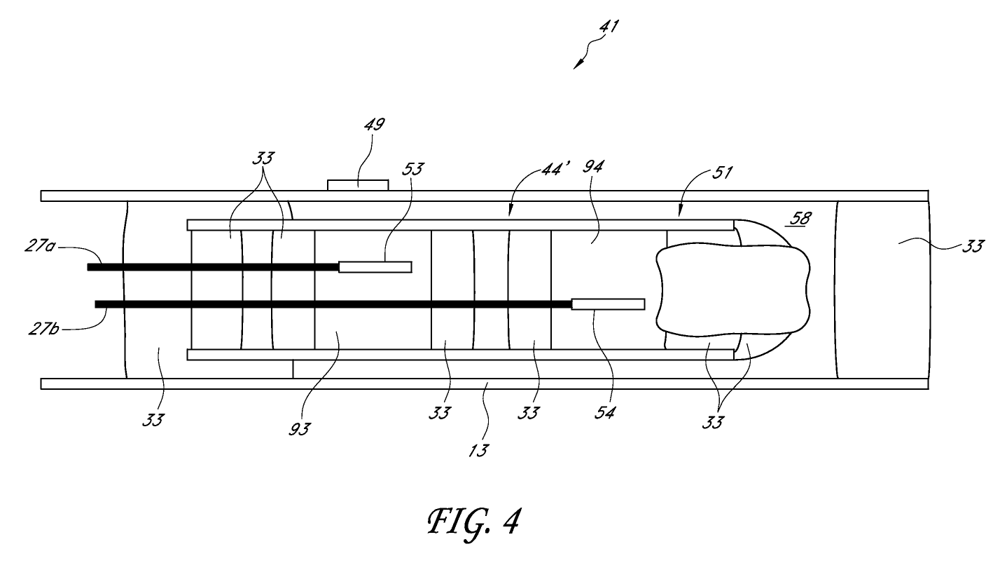

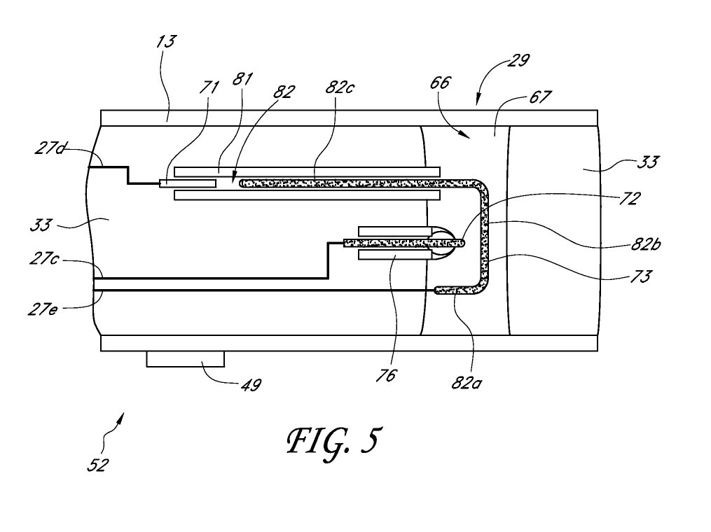

At least one sensor, but, in some embodiments, a plurality of sensors is carried by distal extremity 14b of cannula 13 in the sensor section 24 of probe 12. The sensor section 24 of the probe 12 includes electrodes inside at least one electrolyte-filled chamber. Such multiple sensors can include a carbon dioxide sensor 41, an oxygen sensor 42, a pressure sensor 43 and a pH-sensing electrode 44, or any combination thereof or other sensors. Some or all of such sensors can be utilized for determining gas characteristics of the blood in a vessel of a mammalian body. The carbon dioxide sensor 41 and the oxygen sensor 42, separately or combined, are sometimes referred to herein as a gas sensor assembly. In some embodiments, at least the portion of the cannula or sleeve 13 that is placed inside the blood vessel, including the sensor section 24, is provided with a surface treatment 49, a portion of which is shown in FIGS. 4 and 5, to inhibit the accumulation of thrombus, protein or other blood components which might otherwise impair the blood flow in the vessel or impede the diffusion of oxygen or carbon dioxide into the chambers of the sensor section 24.

The individual sensors of sensor section 24 each occupy a small axial length of the probe 12, for example in the range of five to ten millimeters and, in some embodiments, approximately six millimeters, so that the sensor section 24 of the probe 12 is relatively short, such as less than 25 millimeters, to be easily advanced into a tortuous vessel.

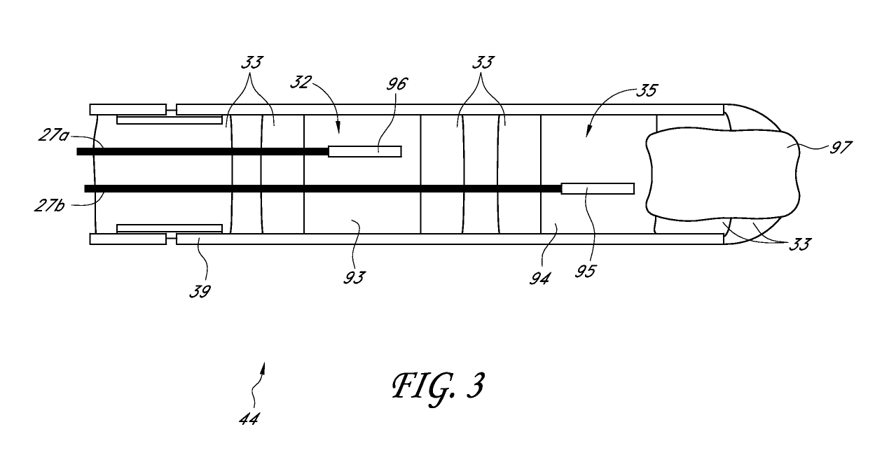

The pH sensor 44, shown in detail in FIG. 3, is carried by distal extremity 14b of the cannula 13 and contained within the sensor section 24 of probe 12. As shown in FIG. 3, there are two cells: the potential of is dependent on the pH of the blood surrounding probe 12 (the working, or pH sensing, cell) and the reference cell provides a reference potential (the voltage reference cell). The pH sensor 44 functions like any classic pH sensor, that is, the pH-sensing electrode 96 is of sufficient area to generate a measurable pH-dependent potential. The voltage reference electrode 95 generates a potential that is essentially independent of pH. Measurement of the potential of the pH-sensing electrode 96, with respect to the potential of the voltage reference electrode 95, allows quantification of the pH of the blood that is in contact with the frit 97 and the external surfaces of the walls of the chamber surrounding pH-sensing electrode 96.

The two cells are separated from each other and from the rest of the sensors in probe 12 by insulating walls; each insulating wall consists of one or more layers of insulators, such as adhesive encapsulant 33, encapsulated air, and/or other material.

The most distal cell of pH sensor 44 is the voltage reference cell and consists of the aforesaid walls, a chamber 94, an electrolyte solution or conductive gel N01 filling chamber 94, a reference electrode 95 which is immersed in this solution or gel, and a frit 97. The electrode 95 can be formed from a silver wire which is coated with silver chloride at its distal end, produced by dipping the silver wire into molten silver chloride, or alternatively by a known electrochemical process. The cylindrical wall of chamber 94 is of any material such as glass or plastic which is relatively impermeable to gases in the blood. Embedded in the adhesive encapsulant 33 which seals the distal end of chamber 94 is a frit 97, composed of an appropriate porous material such as ceramic or glass, such as Vycor 7930. The distal end of frit 97 is exposed to blood; the proximal end of frit 97 is exposed to the electrolyte solution or conductive gel which fills chamber 94. The properties of the frit enable and house a liquid junction between the blood on the outside of probe 12 and the solution or gel which fills chamber 94.

The pH-sensing cell of pH sensor 44 is just proximal to the voltage reference cell, separated as mentioned by one of the aforesaid insulating walls. The pH-sensing cell consists of the aforesaid insulating walls, a chamber N02, a pH buffered solution N03 filling chamber N02, a pH-sensing electrode 96, and cylindrical walls N04 that are composed of pH sensitive glass. The pH-sensing electrode 96 is formed in the same way that the voltage reference electrode 95 is, and is immersed in the pH buffered solution filling chamber N02.

The pH-sensing electrode 96 is attached to an electrical conductor 27g, such as an insulated copper or platinum wire, by soldering or welding. The portion of conductor 27g extending from electrode 96 through chamber N02 and back to the connector 17 is covered with any suitable insulating material. The voltage reference electrode 95 is attached to an electrical conductor 27h, such as an insulated copper or platinum wire, by soldering or welding. The portion of conductor 27h extending from electrode 95 through chamber 94 and chamber N02 and back to the connector 17 is covered with any suitable insulating material. Alternatively, the conductors 27g and 27h are silver wires, the distal ends of which are formed into electrodes 96 and 95, respectively.

A detailed view of the carbon dioxide sensor 41 contained within the sensor section 24 of probe 12 is shown in FIG. 4. The carbon dioxide sensor 41 consists of a smaller embodiment of pH sensor 44, called herein the carbon-dioxide-sensing-element, which is suspended in a chamber 51. The adhesive encapsulant 33 seals each end of chamber 51 and secures the proximal end of the carbon-dioxide-sensing-element. The chamber 51 is preferably filled with an electrolyte solution 58 such as a mixture of 0.154 Molar NaCl (normal saline) and 0.026M NaHCO (sodium bicarbonate). The cells, electrodes and conductive elements for the carbon-dioxide-sensing-element are made with the same methods as the cells, electrodes and conductive elements for the pH sensor 44. Conductors 27a and 27b are connected to the sensing electrode 53 and the reference electrode 54, respectively, of the carbon dioxide sensor 41 in the same way that their counterparts are connected to the electrodes of the pH sensor 44.

As with the pH sensor 44, the pH-sensing cell of carbon-dioxide-sensing-element generates a measurable pH-dependent potential and the voltage reference cell generates a potential that is essentially independent of pH. Carbon dioxide gas permeation through the polymethylpentene membrane of the cannula 13 of the present embodiment results in a pH change in the electrolyte solution 58 which in turn causes a change in potential of the pH sensing cell. This change in potential is proportional to the carbon dioxide partial pressure in the blood surrounding probe 12. Measurement of the potential of the pH-sensing cell of carbon-dioxide-sensing-element, with respect to the potential of the voltage reference cell of carbon-dioxide-sensing-element, allows quantification of the carbon dioxide partial pressure in the blood outside the probe 12.

The oxygen sensor 42 is illustrated in FIG. 5 and can include an oxygen main chamber 66 containing an electrolyte solution 67, a first or reference electrode 71, a second or working electrode 72 and a third or counter electrode 73. The main chamber 66 is defined by the cannula or sleeve 13 and the adhesive encapsulant 33, which seals each end of the chamber. The main chamber 66 is preferably filled with the electrolyte solution 67, such as 0.154 Molar NaCl (normal saline).

The cathode or working electrode 72 extends through a first tube 76 made from any suitable nonconductive insulating material such as polyimide and, for example, having an outer diameter of 0.005 inch, an inner diameter of 0.004 inch and a length of 8 mm. The cathode or working electrode 72 is formed by exposing a small portion of bare platinum wire to the electrolyte solution 67 in main chamber 66. This cathode or working electrode 72 protrudes slightly from an encapsulant of either sealing glass or an insulating adhesive. If sealing glass is used, a bead of sealing glass is fused near the distal end of the bare portion of the platinum wire so that the wire extends through the glass bead, near the center, protruding beyond the glass bead. The platinum wire diameter can range from 0.001 inch to 0.004 inch, and, in some embodiments, is 0.002 inch, and protrudes from 0.1 to 0.3 mm beyond the encapsulant or the bead of sealing glass. The non-protruding portion of the platinum wire is contained in tube 76. The protruding portion of working electrode 72 is preferably rounded and smoothed, by some means such as laser melting. The purpose of this rounding and smoothing is to ensure there are no sharp edges or splinters to cause unwanted irregularities in the electric field potential around the tip of working electrode 72.

The proximal end of the working electrode 72 is attached or otherwise coupled to a third electrical conductor 27c, for example by soldering or welding. Alternatively, and preferably, working electrode 72 and electrical conductor 27c are the same platinum wire, and the working electrode 72 is formed by stripping the insulation from electrical conductor 27c at the distal tip. The first tube 76, and the proximal portion of the glass bead,are embedded within the adhesive encapsulant 33, which additionally seals the proximal end of the first tube 76 as well as sealing the glass bead to the first tube 76. The bare distal end of the working electrode 72 is situated in and exposed to the electrolyte solution 67 within oxygen main chamber 66.

The reference electrode 71 of the oxygen sensor 42 can be formed from a silver wire coated with silver chloride, for example, by dipping the silver wire into molten silver chloride or alternatively by any suitable electrochemical process. The electrode 71 has a diameter ranging from 0.001 inch to 0.003 inch and preferably approximately 0.002 inch. The sensor 42 further includes a second tube 81 made from any suitable nonconductive material such as plastic and preferably a polymer. The second tube 81 extends along the first tube, substantially parallel to the first tube, and is provided with an internal bore 82. Tube 81 can have an outer diameter of 0.004 to 0.006 inch, preferably 0.005 inch, an inner diameter of 0.003 inch to 0.005 inch, preferably 0.004 inch, and a length of 3 to 8 mm. In some embodiments the length of the second tube is 5 mm. As can be seen, the inner diameter of the second tube 81 is only slightly larger than the outer diameter of the reference electrode 71. Substantially the entire length of the second tube is secured or embedded in the polymer adhesive or adhesive encapsulant 33. The internal bore 82 of the second tube 81 is free of the adhesive encapsulant 33 except at its proximal end; the distal opening of the second tube 81 communicates with main chamber 66 so that solution 67 fills second tube 81 as well as main chamber 66. The proximal end of reference electrode 71, inserted into the proximal end of the second tube, is secured to a conductor 27d by any suitable means such as welding or soldering. Alternatively, electrode 71 and electrical conductor 27d are the same silver wire, and the reference electrode 71 is formed by stripping the insulation from electrical conductor 27d along the distal portion and coating the stripped portion with silver chloride, as described above. The reference electrode 71 extends distally into the second tube 81, in some embodiments extending along the axial centerline of the second tube 81, and the base of reference electrode 71 is bonded to second tube 81, at the same time sealing the proximal end of second tube 81.

Counter electrode 73 can be made from any suitable conductor and can be formed from a platinum wire having a diameter ranging from 0.001 inch to 0.004 inch and approximately 0.002 inch. Electrode 73 has a first or proximal portion 82a electrically coupled to a conductor 27e by any suitable means such as soldering or welding. Alternatively, electrode 73 and electrical conductor 27e are the same platinum wire, and the electrode 73 is formed by stripping the insulation from electrical conductor 27e along its distal portion. The proximal portion 82a extends along the first tube 76, and can be parallel to the tube 76 and on the opposite side of the first tube from second tube 81. Electrode 73 has a second or central portion 82b that forms a curve or loop that extends over to second tube 81, so as to pass near the working electrode 72. This central portion 82b is disposed in oxygen main chamber 66; the center of the loop of electrode 73 is spaced 0.1 to 0.5 mm, in some embodiments 0.25 mm, from the working electrode 72. The electrode 73 is further provided with a third or distal portion 82c that is parallel to the proximal portion 82a, and extends into the distal opening of the second tube 81 and through much of the second tube 81. Proximal portion 82a, central portion 82b and distal portion 82c of electrode 73 are stripped bare of insulation.

The tips of reference electrode 71 and counter electrode 73 are contained within second tube 81 and close to each other, but not touching, and in this regard are separated by a distance ranging up to and including 1.5 mm and which can be approximately 1 mm. The opposed tips are located a considerable distance from the distal opening of second tube 81, and in this regard the counter electrode 73 extends proximally into the second tube 81 a distance ranging from 3 to 7 mm. In some embodiments, the counter electrode 73 extends proximally into the second tube 81 a distance of approximately 5 mm. The tip of counter electrode 73, which is near reference electrode 71, is rounded and smoothed in the same manner as the tip of working electrode 72.

Oxygen gas permeation through the polymethylpentene membrane of cannula 13 of the present embodiment results in a change in the oxygen concentration in the electrolyte solution 67. Electronic circuitry (not shown) within display module 11 maintains the desired potential of 0.70 volts between the working electrode 72 and the reference electrode 71 while measuring the flow of current from the counter electrode 73 to the working electrode 72. The magnitude of this current is proportional to the concentration of O2 in the electrolyte solution 67 within oxygen main chamber 66 which, in turn, is dependent on the partial pressure of oxygen in the blood surrounding the probe 12 at the oxygen sensor 42. The electrochemical reaction at the working electrode 72 can be described as:

O₂(g)+2H₂O+4e⁻→4OH—

The reaction at the counter electrode 73 is believed to be the reverse of this. At the reference electrode 71, the reaction can be described as:

Ag(s)+Cl—→AgCl(s)+e−

Migration of positively charged silver ions (Ag+) to working electrode 72 is inhibited by placing the end of the counter electrode 73 close to, but not in contact with, the opposed end of the reference electrode 71 so as to provide a positive electric field in the vicinity of the reference electrode 71 to repel Ag+ ions and by placing the counter electrode 73 and reference electrode 71 in second tube 81, which has a relatively narrow diameter, thus reducing the migration rate for Ag+ ions to working electrode 72. In an alternative embodiment, such migration is further inhibited by replacing some or all of the electrolyte in the second tube 81 or in the main chamber 66 with the conductive gel, separating the reference electrode 71 from the main volume of electrolyte solution 67 disposed in the oxygen main chamber 66, and thus further reducing the migration rate of Ag+ ions to working electrode 72. In general, inhibiting the migration of positively charged silver ions to working electrode 72 minimizes any upward drift in the signal from the working electrode caused by silver deposition on the working electrode.

In an alternate embodiment of oxygen sensor 42, shown in FIG. 5A, a large reference chamber N05 is formed by distal and proximal walls of adhesive encapsulant and the cylindrical walls of cannula 13. The inner diameter of large reference chamber N05 matches that of cannula 13. The distal adhesive wall of large reference chamber N05 is positioned distal to and very near the proximal end of second tube 81 but in such a way that the adhesive does not enter second tube 81. The proximal adhesive wall of large reference chamber N05 is placed some distance from the distal adhesive wall of large reference chamber N05, at least far enough to accommodate a useful length of the reference electrode 71, which can be, in some embodiments, 1 mm.

In the embodiment of FIG. 5, the tip of counter electrode 73 is near the proximal end of second tube 81, preferably emerging slightly from second tube 81. The reference electrode 71 can be placed anywhere in large reference chamber N05, as long as it does not touch counter electrode 73. The purpose of large reference chamber N05 is to reduce the likelihood of a gas bubble blocking the proximal opening of tube 81 and the path of conductive ions between large reference chamber N05 and main chamber 66.

Although occupying a small axial length of the probe 12, oxygen sensor 42 maintains a large physical separation between the working electrode 72 and the reference electrode 71, provides a large volume of electrolyte solution, and inhibits the migration of silver ions to the working electrode 72 and thus the buildup of silver precipitate on the working electrode 72. Additionally, only a small and well-defined surface area of the working electrode 72 is exposed to the electrolyte solution 67.

As can be seen in the embodiment of FIG. 1, the cylindrical cannula or sleeve 13 of gas permeable material forms a large surface area circumferential window 29 for both the carbon dioxide sensor 41 and the oxygen sensor 42. Such a circumferential window 29 is particularly advantageous as the covering for the blood gas sensor chambers 51 and 66 since it maximizes the permeable membrane area for a given sensor length. In addition to maximizing the permeable membrane area, the circumferential window 29 eliminates the “wall effect” artifact wherein the gas permeable membrane on the tip or one side of a blood gas sensor probe is fully or partially blocked from exposure to the blood when the probe is inadvertently positioned against a vessel wall. Since the functionality of the carbon dioxide and oxygen sensors is primarily affected by the ability of the gas in the blood to reach equilibrium with the solution in the gas sensor chamber, even if the probe is inadvertently placed against a vessel wall, the circumferential window will assure that a gas permeation path into the sensor chambers 51 and 66 still exists so that equilibrium is achieved. Therefore, the sensitivity of the oxygen sensor 42 and carbon dioxide sensor 41 to the wall effect artifact is minimized by having a circumferential window comprised of a membrane material that is as highly permeable to oxygen and carbon dioxide gases as possible.

Additionally, in some embodiments, both the carbon dioxide and oxygen sensors function so that they do not continuously consume reactants (such as electrolyte or gas) during their operating lifetime.

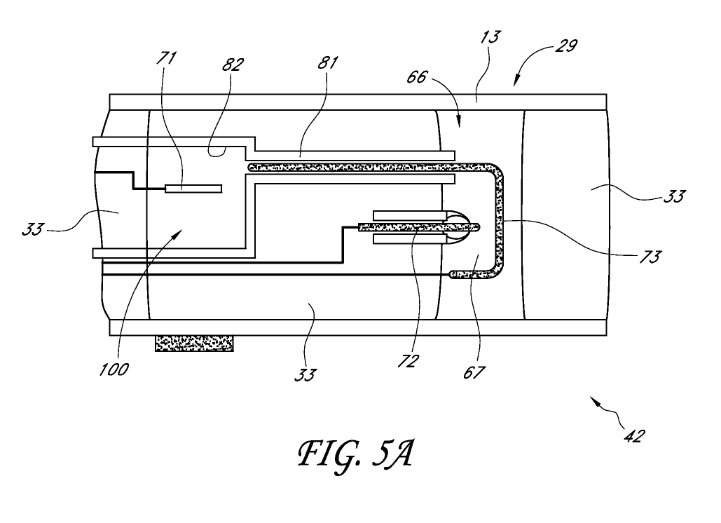

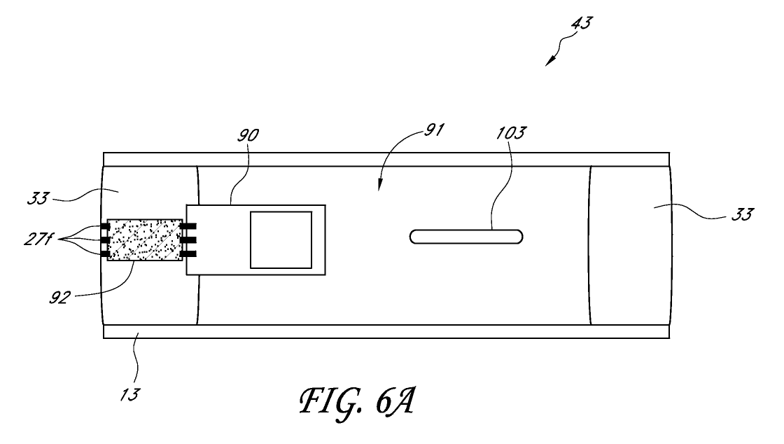

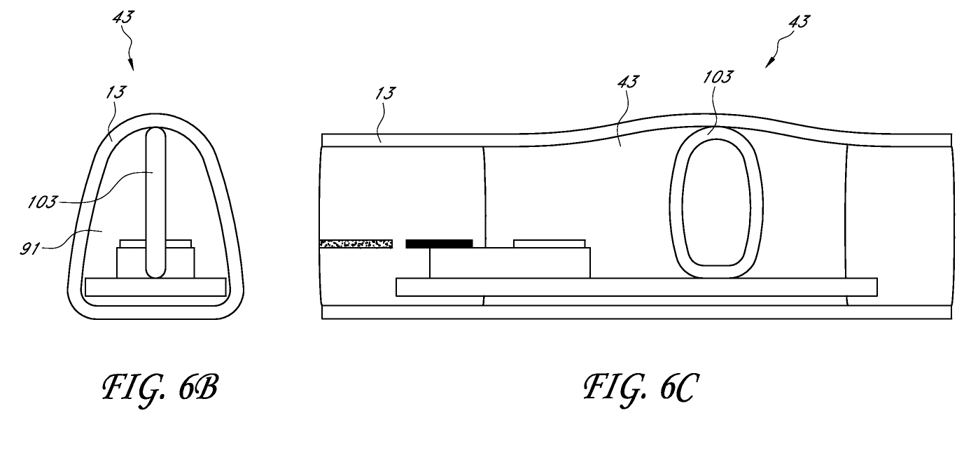

The distal extremity 14b of cannula 13 is further provided with a pressure sensor 43, shown in FIG. 6A. This sensor 43 can in principle be placed either proximal to, or distal to, either the oxygen or carbon dioxide gas sensor chambers. The pressure sensor chamber 91 is sealed on either end from other chambers with the adhesive encapsulant 33. The connector end of the pressure sensing element 90 is embedded in the proximal encapsulant 33 in order to insulate the connector pads and maintain the placement of the pressure sensor 43 in the chamber 91. The sensing portion of pressure sensing element 90 extends into pressure chamber 91, and is immersed in the fluid filling chamber 91. The diaphragm of the pressure sensing element 90 is fully within the chamber 91, with no part of it touching the adhesive encapsulant 33. This allows it to respond fully to changes in pressure in chamber 91.

The pressure sensing element 90 is appropriately small in size and, for example, can have a length ranging from 0.020 to 0.100 inch and preferably approximately 0.060 inch, a width ranging from 0.010 to 0.015 inch and preferably approximately 0.012 inch and a height ranging from 0.010 to 0.015 inch and preferably approximately 0.012 inch. The length and width and height of the pressure sensing element 90 are visible in FIGS. 6A and 6B.

The pressure sensing element 90 can be of any suitable type, such as of the solid state type manufactured by Silicon Microstructures of Milpitas, Calif. The pressure sensing element 90 is preferably a piezoresistive silicon sensor and, for example, can be a two-resistor, or half-bridge, design using three lead wires. Alternatively, the pressure sensing element 90 is a four-resistor, full-bridge, design using four lead wires. The isolation of the pressure sensing element 90, for example in its own chamber 91, can be advantageous because it cannot function in an ionic solution without a special insulative coating which would dampen its sensitivity. Its chamber 91 is filled with a non-conductive fluid such as silicone oil.

A plurality of conductors 27f extend from the pressure sensing element 90 to respective electrical contacts 18 provided in probe connector 17 to permit electrical communication with the sensor 43 from the proximal extremity of the probe 12. In a preferred embodiment, the conductors 27f are contained within a cover 92. The cover 92 is made from any non-conductive flexible material such as plastic and is optional; it is provided solely to make the assembly process simpler.

In order to facilitate desirable transduction of the vessel pressure surrounding the cannula 13 at pressure sensor 43, the effective stiffness of the cannula should be a small fraction of the stiffness of the silicon diaphragm of the pressure sensing element 90. A relatively large area of the cannula relative to the sensor diaphragm and a low modulus of elasticity of the material of the cannula relative to the silicon material of the sensor diaphragm contribute to the effective stiffness of the cannula 13 being a small fraction of the stiffness of the diaphragm of the sensor 43. The stiffness of the wall of the cannula 13 should be low enough that it does not significantly impede the transduction of a pressure change in the bloodstream to the diaphragm of pressure sensing element 90.

In addition, in some embodiments, the cross-section of the cannula 13, in the region of the pressure sensor chamber 91, is not perfectly round, but is, for example, oval. A mechanism for causing this shape is shown in FIGS. 6B and 6C and consists of a stretcher consisting of a loop (shown) or a block or plug of some non-conductive material to force cannula 13 to be out-of-round in much of the chamber 91. This helps ensure that the round shape of cannula 13 does not resist a pressure change, but transmits it to a large degree to the fluid filling chamber 91, which in turn transmits the pressure change to the diaphragm of the pressure sensing element 90.

In one embodiment, the pressure sensing element 90 is capable of additionally serving as a temperature sensor, although it is appreciated that any other separate thermocouple, thermistor or other pressure sensor can be provided. If needed, the placement of a separate temperature sensor in close proximity to carbon dioxide sensor 41 and oxygen sensor 42 permits the temperature sensor to accurately reflect the temperature of the surrounding blood.

As discussed above, the sleeve or cannula 13 provides a substantial portion of the probe strength, particularly in the sensor section 24, where the sensor chambers 51, 66, 91, 94 and NN are filled with liquid.

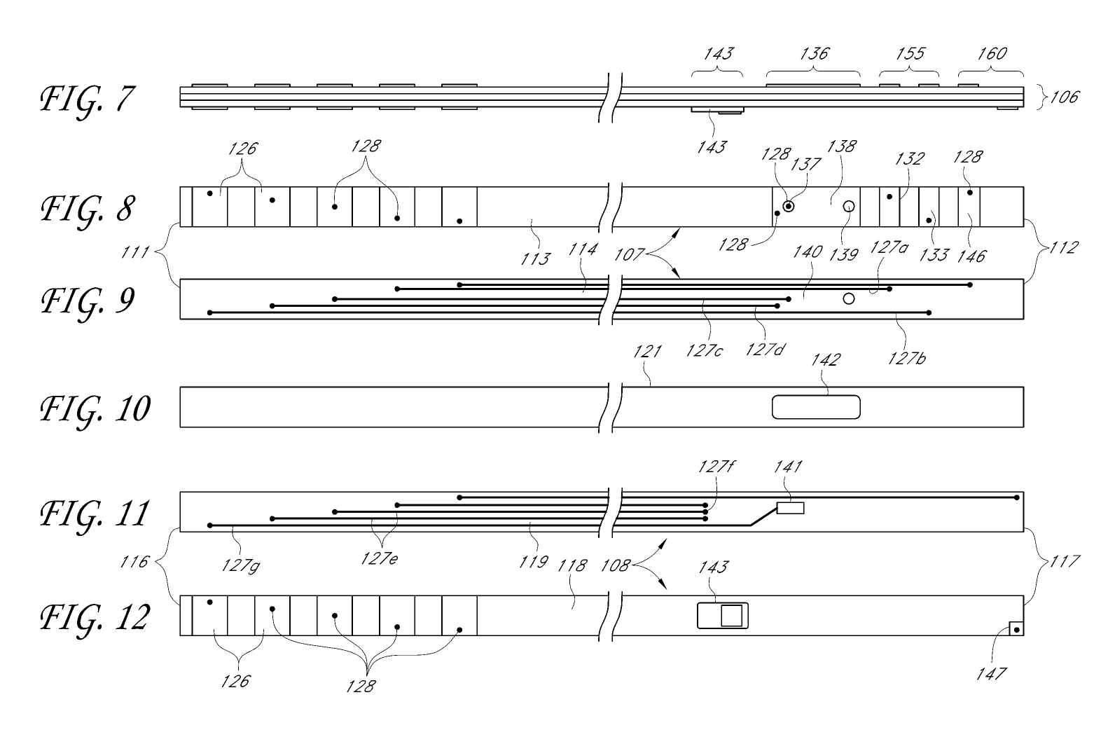

In another embodiment of the probe of the present invention, illustrated in FIGS. 7-12, the various internal wires, conductors and sensors disclosed above with respect to probe 12 can be wholly or partially replaced with a flexible printed circuit assembly 106 formed from a plurality of layers of a nonconductive substrate. The flexible printed circuit assembly 106 has a length, such as 25 centimeters, appropriate for the assembly to be situated longitudinally within the lumen of a sleeve, such as cannula 13, and has a width ranging from 0.008 to 0.017 inch and preferably 0.015 inch. More specifically, assembly 106 is formed from first, second and third layers 107, 121 and 108 of a suitable insulating material such as polyimide. First layer or flexible substrate 107 has proximal and distal extremities 111 and 112 and a first or outer planar surface 113 and a second or inner planar surface 114. Similarly, third layer or flexible substrate 108 has proximal and distal extremities 116 and 117 and a first or outer planar surface 118 and a second or inner planar surface 119. Second layer 121 specifically engages the inner surfaces 114 and 119 of the layers 107 and 108, while providing electrical and mechanical isolation of inner surfaces 114 and 119 from each other.

A plurality of contact pads 126 are formed on the proximal extremities of the first and third layers 107 and 108 for forming a low profile connector similar to connector 17 of probe 12. In this regard, and as shown in FIG. 8, a plurality of five contact pads 126 are formed on outer surface 113 of the first layer 107. As shown in FIG. 12, a plurality of five contact pads 126 are formed on outer surface 118 of third layer 108. A plurality of electrodes are formed on the distal portion of the flex circuit assembly 106 and a plurality of conductive traces or conductors 127 are formed on the layers 107 and 108 for electrically coupling the contact pads 126 to respective electrodes. More specifically, and as shown in FIG. 9, a plurality of five conductors 127 extend longitudinally from the proximal extremity 111 to the distal extremity 112 along the inner surface 114 of first layer 107. A plurality of five conductors 127, as shown in FIG. 11, extend longitudinally from the proximal extremity 116 to the distal extremity 117 along the inner surface 119 of third layer 108. As such, the conductors 127 are sandwiched or disposed between the first and third layers 107 and 108 and the insulating second layer 121. The conductors 127 on first and third layers 107 and 108 are electrically connected to respective contact pads 126 by feedthrough vias 128 extending between the outer and inner surface of each of the layers 107 and 108.

The plurality of sensors carried by the distal extremity of the flex circuit assembly 106 includes one or more of a pH sensor NN, a carbon dioxide sensor NN, an oxygen sensor 136, and a pressure sensor 143.

A pH sensor assembly, as described in FIG. 3, is attached to contact pads 146 and 147. Contact pad 146 is provided on outer surface 113 of first layer 107 and electrically connected to conductor 127e by means of a via 128. Contact pad 147 is provided on outer surface 118 of third layer 108 and electrically coupled to a conductor 127g on inner surface 117 by means of a via 128.

A carbon dioxide sensor NN, as described with respect to FIG. 4, is attached to contact pads 132 and 133, which are formed on outer surface 113 of first layer 107. Contact pad 132 is electrically coupled to conductor 127a on inner surface 114 by means of via 128 and contact pad 133 is electrically coupled to conductor 127b on inner surface 114 by means of a via 128.

An oxygen sensor 136 is additionally provided, as part of the flex circuit layout, and includes a working electrode pad 137 formed on the outer surface 113 of first layer 107 (FIG. 8) and electrically coupled to conductor 127d (FIG. 9) by means of via 128. The sensor 136 includes a counter electrode pad 138 formed on outer surface 113 and electrically coupled to conductor 127c by means of via 128. Thus the working electrode pad is encircled by, but not connected directly to, the counter electrode pad 138. The counter electrode pad 138 is electrically coupled by via 139, extending between the surfaces 113 and 114, to an electrode pad 140 on surface 114. Thus, the counter electrode in oxygen sensor 136 consists of electrode pads 138 and 140 and via 139. A reference electrode pad or reference electrode 141 is included in oxygen sensor 136 and is formed on the inner surface 119 of third layer 108. The reference electrode pad 141 is electrically coupled to conductor 127g. Second layer 121 has a cutout 142 that provides the boundaries of a shallow chamber; the top of this chamber is covered in part by counter electrode pad 140 and the bottom of this chamber is covered in part by reference electrode pad 141. Via 139 is large enough, preferably 0.003 inch in diameter, so that when the three layers 107, 108, and 121 are assembled and the assembly is inserted into a cannula or sleeve as discussed below and electrolyte solution such as 67 is introduced into the cannula or sleeve, the electrolyte solution such as 67 can easily fill this chamber as well as the volume surrounding oxygen sensor 136.

Flex circuit assembly 106 further includes a pressure sensor 143, preferably including a solid state pressure sensing element like pressure sensor 43 above, mounted on outer surface 118 of third layer 108 and electrically coupled to three conductors 127f on inner surface 119 by means of three vias 128. As discussed above, pressure sensor 143 preferably includes a temperature sensor.

The flexible circuit assembly 106 can be mass-produced in a batch process at low cost, thereby minimizing the cost of the multi-sensor probe. In such a batch process, successive layers of conducting materials on insulating substrates, that is layers 107 and 108, are deposited by electroplating, vapor deposition or other methods, then they are patterned by photolithography, laser ablation or other methods. The pads forming contact pads 126 and the various sensors and the traces or conductors 127 of the flexible circuit assembly 106 are primarily formed of copper. The pads are plated with various metals including silver, platinum and gold to create the electrodes of the various sensors or contact pads for attaching the carbon dioxide sensor, the pH sensor and the blood pressure sensor. The contact pads 126 are plated with gold to provide reliable electrical contact with the mating connector of the display module 11. The contact pads 132 and 133 are plated with gold to provide reliable surfaces for attaching a carbon dioxide sensor 41. The working electrode 137 for the oxygen sensor 136 is preferably formed by masking a platinum-plated pad electrode with an insulating material to define a small exposed area of platinum metal in the range from 0.001 to 0.008 inch in diameter and preferably approximately 0.002 inch in diameter. The reference electrode 141 for the oxygen sensor is electrochemically plated with silver chloride.

The contact pads 146 and 147 are plated with gold to provide reliable surfaces for attaching a pH sensor. In addition to or as an alternative to the temperature sensor in pressure sensor 143, the flexible circuit assembly 106 can support a temperature sensor in the form of a patterned thin film of known material forming a temperature-sensitive resistor on the inner surface of one of the layers 107 and 108, or the temperature sensor can be a diode, thermistor, or thermocouple bonded to one of the flexible circuit layers 107 and 108. The patterned layers 107, 121 and 108 are bonded together with insulating adhesive to complete the multi-layer flexible circuit assembly 106.

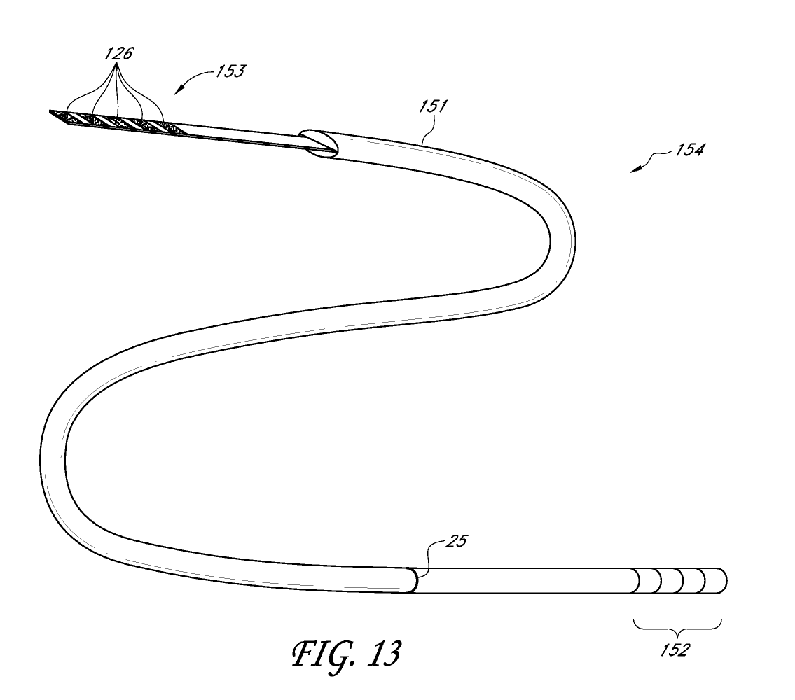

Once the processing steps have been completed from sheets of substrate materials that have been patterned and adhered in the manner discussed above, individual circuit assemblies are cut from the sheets. The individual circuit assemblies are thus formed into narrow strips, for example having a width of 0.015 inch, such that each circuit assembly 106 can be inserted into a cannula or sleeve 151, substantially similar to cannula or sleeve 13, and filled with an adhesive encapsulant 33 and electrolyte solutions or other liquids of the type discussed to form the sensor chambers 94, 51, 66, NN and 91 in the sensor section 152 of the flexible circuit assembly 106. FIG. 13 illustrates a flexible circuit assembly 106, including various electrodes such as sensors 131, 136, 143 and 147, inserted into the lumen or bore of the cannula or sleeve 151. The proximal end or portion of the flexible circuit 106 includes buried traces or conductors 127 and gold-plated pads 126 which serve as conductors and contacts for the low profile connector 153 of probe 154, which is much like low profile connector 17 discussed above. The buried traces conduct electrical signals from the sensor electrodes or sensor pads to the electrical contacts pads 126, which serve as a low profile electrical connector 153 that can be coupled to the mating connector 166 of the display module 11.

As described above, at least the portion of the polymer cannula or sleeve 13 or 151 that forms the external surface of the respective probe is preferably provided with a durable surface treatment 49, a portion of which is shown in FIGS. 4 and 5, to inhibit the accumulation of thrombus, protein, or other blood components, which might otherwise impair blood flow in the artery or impede the transport of oxygen or carbon dioxide through the circumferential window 29 into the sensing chambers 51 and 66. One preferred method for treating the surface of the cannula or sleeve 13 or 151 is photoinduced graft polymerization with N-vinylpyrrolidone to form a dense multitude of microscopic polymerized strands of polyvinylpyrrolidone, covalently bonded to the probe outer surface. This surface treatment 49 is durable, due to the strong covalent bonds, which anchor the polymer strands to the underlying substrate. Procedures for surface treatment of the polymer cannula or sleeve material are described in copending application Ser. No. 10/658,926 filed Sep. 9, 2003, which is hereby incorporated by reference in its entirety as if set forth fully herein.

The surface treatment 49 adds only a sub-micron thickness to the probe body 13 or 151, yet it provides a hydrophilic character to the probe surface, rendering it highly lubricious when hydrated by contact with blood or water, thereby facilitating the smooth passage of the probe 12 or 154 through the blood vessel. This hydrophilic surface treatment 49 also inhibits the adsorption of protein onto the surface of the underlying polymer substrate, thereby minimizing the accumulation of thrombus, protein, or other blood components on the probe. Although the dense multitude of polyvinylpyrrolidone polymer strands shields the underlying outer wall of the sleeve or cannula from large protein molecules, it does not significantly impede the migration of small molecules such as oxygen or carbon dioxide through the wall of the cannula. Therefore, the surface treatment 49 of the polymethylpentene cannula or sleeve 13 or 151 facilitates consistent, reliable communication of the gases in the blood, such as oxygen and carbon dioxide, through the circumferential window 29 into the carbon dioxide and oxygen sensor chambers 51 and 66, even after prolonged residence time up to three days in the bloodstream of a patient.

The display module 11, as shown in FIG. 1, includes a housing 161 formed of a suitable material such as plastic and which is sized so that it can be worn on the patient, such as on the patient's wrist, arm or other limb, sometimes referred to herein as the subject, with the probe 12 or 154 inserted into vessel(s) in the hand, wrist, forearm or other peripherally accessible vessel. The module 11 also includes a display 162 such as a liquid crystal display (LCD) for displaying measured parameters and other information, and adapted to be readily visible to the attending medical professional, sometimes referred to herein as the user. The display 162 may include backlighting or other features that enhance the visibility of the display. A band 163 attached to the housing 161 is adapted to secure the display module 162 to the subject's wrist. Alternatively, the module 11 may be attached to the subject's arm or to a location near the subject. Optionally, in the case the subject is a newborn infant (neonate), the module 11 may be strapped to the subject's torso, with the probe 12 or 154 inserted into umbilical vessel(s). The band 163 is comprised of any suitable material, such as Velcro or elastic. Buttons 164 or keys facilitate entry of data and permit the user to affect the display 162 and other features of the module 11. While FIG. 1 shows three buttons, any number or type of buttons, keypads, switches or finger-operable elements may be used to permit entry of parameters or commands, or to otherwise interface with the apparatus 10. Alternatively, there may be no buttons for affecting the display 162, in which case the various screens 162 would appear automatically, in sequence one after the other, at a rate consistent with medical practice. For example, each screen 162 might appear for 3 seconds before it was replaced by the subsequent screen. The module 11 may also include wireless communications capability to facilitate display of physiologic parameters on a remote monitor or computer system, and/or to facilitate the entry of patient parameters or other information into the module 11 from a remote control panel or computer system. The module 11 also includes one or more connectors 166 that provide physical connection and communication with one or more probes 12 or 154. Preferably, each connector 166 includes a receptacle adapted to receive, secure, and communicate with a corresponding connector 17 or 153 on the proximal end of the respective probe 12 or 154.

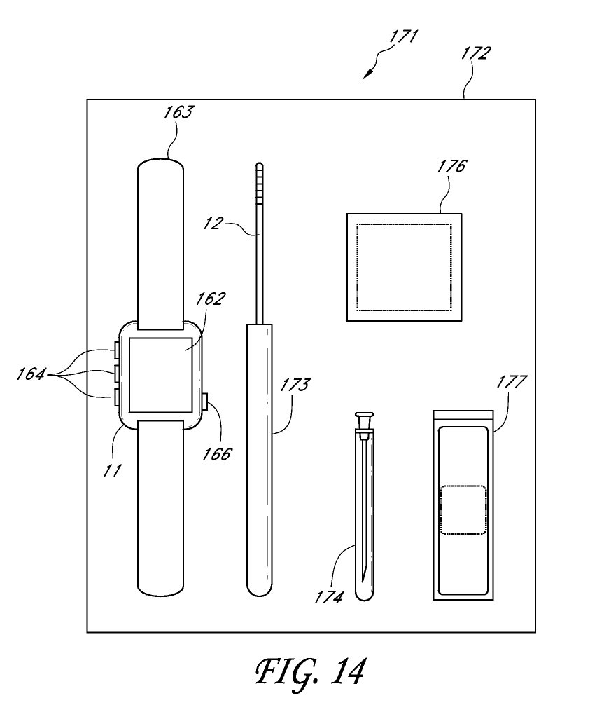

In a preferred embodiment of the display module 11, the module is designed to be low in cost so that it can be packaged together with one or more probes 12 or 154 and accessories as a disposable kit 171, with all of the components of the kit packaged together in a sterile pouch or other container 172, as illustrated in FIG. 14. In addition to the display module 11 and one or more probes 12 or 154, the kit 171 would optionally include a probe holder 173 to protect the probe from damage or degradation, a wrist band 163 or other means for attaching the display module to a patient, a needle or other introducer 174, alcohol swabs 176 for cleaning the skin prior to cannulating the vessel and for cleaning blood or other residue from the probe connector prior to attaching the probe to the module, a bandage 177 to cover the puncture site and anchor the probe in place, and any other items that may be utilized for preparing and using the probe and display module 11. The display module 11 is further designed to require low power so that it can operate for the expected lifetime of the device, such as 72 hours, on battery power without the need for battery replacement or connection to an external power source.

Each of the probes 12 and 154 is preferably suited to be a single-use, disposable device, since it has a limited operational lifetime and is used in direct contact with the subject's blood. The module 11 is durable enough to be used many times, however, the advantage of a disposable module is that it eliminates the expense and the infection hazard associated with cleaning, replacing batteries, and reusing a single module for multiple patients. An additional advantage of a disposable module 11 packaged together with its associated probe is that the calibration data can be stored in the module at the time of manufacture, greatly simplifying the use of the apparatus 10 by eliminating the need for the user to enter calibration data into the module prior to using the probe. A further advantage of a disposable module 11 packaged together with its associated probe is that the calibration data stored in the module at the time of manufacture can account for all of the monitor and probe inaccuracies and artifacts in a single set of calibration coefficients, thereby avoiding the accumulation of inaccuracies that can occur with separate calibrations of the probe and the module 11.

In a first embodiment of the module, no user inputs at all are required, eliminating the need for buttons, keypads, switches and other finger operable elements. In this embodiment, the different display screens shown in FIG. 1 would be shown alternately in an automatically switched sequence designed to best suit the needs of the users. The display module 11 is automatically energized upon connection of the one or more probes 12 or 154 to the module 11, and all of the calibration data and other needed information is pre-programmed into the module at the time of manufacture. Suitable electronic circuitry are included in the display module 11, such as shown and described in copending U.S. patent application Ser. No. 10/658,926 filed Sep. 9, 2003, for operating the module 11 and the probe coupled thereto. The compact display module 11 makes the most of the wireless communications by freeing the subject from the tubes and cables that normally tether them to their bed, and by eliminating the need for additional bulky instrumentation at the already crowded bedside.

The low profile connectors 17 or 153 are advantageous in this application, since they permit the use of an ordinary hypodermic needle or other suitable introducer 174 to introduce the probe into the blood vessel with minimal trauma to the wall of the blood vessel. The probe 12 or 154 is introduced into the blood vessel by first inserting the appropriately sized hypodermic needle through the skin and into the target vessel. The extremely sharp tip of the hypodermic needle easily penetrates the skin, the underlying tissue, and the vessel wall, while producing minimal trauma. Once the hypodermic introducer needle has entered the target blood vessel, the probe is inserted through the bore of the needle and advanced into the vessel. The blunt tip 26 and the lubricious surface treatment provided on the exterior of cannula 13 or 151 minimize the likelihood of vessel trauma as the probe is advanced within the target vessel. Once the probe is properly positioned within the target vessel, the introducer needle is withdrawn from the artery and the skin, and completely removed from the probe by sliding it off the proximal end of the probe over the low profile connector, leaving the probe in place in the vessel. The low profile connector at the proximal extremity of the probe is connected to connector 166 of the display module 11. During operation, and as shown in FIG. 1 in the first screen of display 162, the arterial blood gas panel that includes oxygen, carbon dioxide, pH, bicarbonate and blood pressure readings can be displayed and thus monitored by apparatus 10. The bicarbonate reading is derived from the circuitry within module 11 from the carbon dioxide and pH readings taken at the sensor section of the probe. Additionally, as shown in the second screen of display 162, shown alongside the module 11, cardiac output, cardiac index, systemic vascular resistance, heart rate and mean arterial pressure readings can be displayed and monitored. Cardiac output is determined from the difference in venous and arterial oxygen concentration. Systemic vascular resistance is determined from cardiac output and blood pressure. The heart rate is the number of heart beats per minute, determined from the data provided by the pressure sensor, and the mean arterial pressure is determined from the systolic and diastolic blood pressure.

In an second embodiment of the display module 11, a minimum number of user input devices are provided so that patient weight, height, hemoglobin and/or hematocrit values can be entered. This will enable the display of cardiac index, as well as a more accurate value of cardiac output.

The small puncture left by the hypodermic needle quickly seals around the body of the probe, thereby preventing excessive bleeding. The puncture site is covered with a bandage 177 and tape to guard against infection and to anchor the probe. Any blood residue on the low profile connector 17 or 153 or the exposed portion of the probe is wiped away with a moist pad or alcohol swab, and the probe connector is then attached to the mating connector 166 on the display module 11. Although the probe of the present invention has been described for use in a blood vessel, it is appreciated that the probe can be introduced into other vessels, lumen or tissue of a body of a patient, by means of any suitable introducer.

From the foregoing it can be seen that the apparatus 10 and method of the present invention makes it possible to measure blood gases and other characteristics of a subject, such as oxygen and carbon dioxide, as well as other blood parameters including temperature, pH and pressure. As hereinbefore described, a single probe may include more than one sensor, e.g., an oxygen sensor, a carbon dioxide sensor, a temperature sensor, a pH sensor and a pressure sensor. The sensors are included in a probe body, for example having a small diameter of less than 0.023 inch so that it can be readily inserted through a 20-gauge needle into a blood vessel in the hand, wrist, or forearm. This probe includes at least one sensor with a window 29 having a large surface area and high permeability to the target gas molecules, which facilitates the rapid diffusion of blood gases into or out of the sensor chamber to ensure a fast response to changes in the blood gas concentration. The probes utilized are preferably blunt tipped and atraumatic to the vessel wall and are preferably provided with an antithrombogenic surface treatment to inhibit the formation of thrombus or the adhesion of protein or other blood components, ensuring consistent performance of the blood gas sensors and minimizing the need for continuous infusion of heparin to maintain a clot-free environment. The probe carries electrical signals from the sensors, through electrical conductors, to a low profile or other connector removeably attached to a mating connector on the display module. The low profile of the preferred connector facilitates the removal of the hypodermic needle or other introducer used to most simply introduce the probe into the lumen of a vein or artery, thereby eliminating the need for using a split sheath introducer or other more complex technique for introducing the probe into the vessel. The display module is small and inexpensive, and it is particularly suited for attachment to the patient's wrist. The apparatus and method herein described may be adapted to the particular requirements of a variety of different medical applications, several of which are outlined below.

For patients in the intensive care unit (ICU) or coronary care unit (CCU), there is typically the need for monitoring arterial blood gases (oxygen and carbon dioxide), pH and blood pressure. Currently, this monitoring is performed on an intermittent basis, typically three to twelve times per day, by drawing a blood sample from an arterial line in the patient's forearm, and delivering the blood sample to a blood gas analyzer. A multi-sensor probe as described herein, providing continuous oxygen, carbon dioxide, pH and pressure measurements, can eliminate the need and the associated expense and risks of placing and maintaining an arterial line and repeatedly drawing blood samples therefrom. Furthermore, the continuous monitoring provided by the present invention gives rapid feedback regarding the effects of any interventions such as adjustments to the ventilator settings or administration of drugs. The timely feedback on the effects of the medical interventions permits the subject to be more quickly weaned from the ventilator and released from the ICU/CCU, a benefit to both the patient and the healthcare system.

In a subset of ICU/CCU patients, where there is a need to monitor cardiac output, the addition of a venous oxygen sensor probe to the previously described multi-sensor arterial probe, makes it possible for the present invention to estimate the cardiac output using a modified arteriovenous oxygen concentration difference equation (the Fick method) as hereinbefore described. Currently, cardiac output is most frequently monitored using the thermodilution technique, which requires placement of a Swan-Ganz catheter in the jugular vein, through the right atrium and right ventricle, and into a branch of the pulmonary artery. The thermodilution technique requires injections of cold saline boluses at intervals, whenever a cardiac output reading is desired. The replacement of the right heart catheter with the present invention greatly reduces the risk to the patient by eliminating the right heart catheterization procedure, and it provides greater utility by providing on demand cardiac output readings without cumbersome injections of cold saline.

In another subset of ICU/CCU patients, where there is a need to frequently monitor cardiac output but not arterial blood gases, a simpler apparatus is a single venous oxygen probe used to monitor the venous oxygen content. This value is combined with independent measurements or estimates of arterial oxygen saturation from a noninvasive pulse oximeter, hemoglobin density from a daily blood sample, and calculated oxygen consumption according to the standard approximation, to calculate cardiac output according to the Fick method. The probe is placed in a vein in the hand, using an experimentally determined compensation factor to account for the expected difference between the oxygen saturation in the right atrium and the oxygen saturation in a vein of the hand. Alternatively, the oxygen probe can be inserted directly through the jugular vein in the neck, into the vena cava or the right atrium of the heart to provide a direct measurement of the oxygen saturation of the mixed venous blood without the need for a compensation factor. Besides its utility for estimating cardiac output, the venous oxygen content is a valuable parameter on its own for assessing the status of the patient.

In neonates, there is frequently the need for arterial and venous blood gas monitoring, along with the measurement of cardiac output and other blood parameters. The present invention is particularly suitable for neonates, since it minimizes if not eliminates the need for drawing blood from the neonate subject with a small blood volume to draw from. The addition of hemoglobin, bilirubin, electrolyte, or glucose sensors to the blood gas and pH and pressure sensors increases the utility of the multi-sensor probe for this application. The probes are conveniently inserted into umbilical arteries and veins, and the display module is appropriate in size to be strapped around the abdomen or other accessible portion of a neonate.

In diagnosing congenital heart defects in neonate and pediatric patients, there is often a need to sample the oxygen saturation in a variety of locations throughout the chambers of the heart and in the great vessels. This oxygen saturation data is normally collected in conjunction with an angiographic study of the heart, and it permits the operation of a malformed heart to be more accurately diagnosed, thereby resulting in more appropriate treatment for the patient. Currently, oxygen saturation data is collected by drawing multiple blood samples through a small catheter from a variety of locations throughout the heart and the great vessels. These blood samples are sequentially transferred to a blood gas analyzer to obtain an oxygen saturation reading for each sample. Using the technology of the present invention, a small oxygen sensor mounted on a probe or guidewire of suitable size such as less than 0.023 inch in diameter and 50 to 150 centimeters in length can be advanced through a guiding catheter to various locations in the heart and the great vessels to sample the oxygen saturation in vivo, thereby reducing the risk to the patient by eliminating the need to draw a large number of blood samples from a small subject and by reducing the time for the procedure.

Although certain preferred embodiments and examples have been discussed herein, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the present disclosure, including the appended claims.

1. A probe for use in a patient having a vessel carrying blood to ascertain characteristics of the blood, the probe comprising:
a cannula adapted to be inserted into a vessel of a patient and having a proximal extremity and a distal extremity, the cannula having a length so that when the distal extremity is in the vessel of the patient the proximal extremity is accessible outside of the patient;
a gas sensor assembly carried within the distal extremity of the cannula for determining gas characteristics of the blood in the vessel;
a pH sensor assembly carried within the distal extremity of the cannula for determining pH of the blood in the vessel; and
a pressure sensor assembly carried within the distal extremity of the cannula for determining pressure of the blood in the vessel.

2. The probe of claim 1, wherein the pressure sensor assembly comprises a solid state pressure sensor.

3. The probe of claim 1, wherein the distal extremity of the cannula comprises a gas permeable material proximate to the gas sensor assembly.

4. The probe of claim 1, further comprising an electrolyte solution disposed within the cannula, and wherein the gas sensor assembly includes electrodes disposed in the electrolyte solution for providing electrical inputs to and electrical outputs from the gas sensor assembly.

5. The probe of claim 1, wherein the gas sensor assembly comprises an oxygen sensor assembly.

6. The probe of claim 5, wherein the gas sensor assembly further comprises a carbon dioxide sensor assembly.

7. The probe of claim 6, further comprising conductor elements connected to the oxygen sensor assembly and to the carbon dioxide sensor assembly and configured to supply electrical outputs to the proximal extremity of the cannula.

8. The probe of claim 5, wherein the oxygen sensor assembly includes:
an electrolyte fill solution disposed in the cannula; and
first, second, and third electrodes disposed in the electrolyte fill solution and configured to provide electrical outputs to the proximal extremity of the cannula.

9. The probe of claim 1, further comprising a flex circuit assembly extending from the proximal extremity of the cannula to the distal extremity of the cannula, wherein the gas sensor assembly, the pH sensor, and the pressure sensor assembly are mounted on the flex circuit assembly.

10. The probe of claim 1, wherein the gas sensor assembly, the pH sensor assembly, and the pressure sensor assembly are attached to conductor elements of a flex circuit assembly that provides a zero profile connector.

11. The probe of claim 2, combined with a module comprising electronics configured to create and digitize analog signals from the pressure sensor assembly and software algorithms configured to provide measurements of systolic and diastolic blood pressure, mean arterial pressure, heart rate, and systemic vascular resistance.

12. The probe of claim 5, wherein the oxygen sensor assembly comprises a working electrode, a counter electrode, and a reference electrode, and wherein the oxygen sensor assembly has a structure that supports high electrochemical activity between the working electrode and the counter electrode and that inhibits electrochemical activity between the working electrode and the reference electrode.

13. The probe of claim 12, wherein a voltage of each of the working electrode, the counter electrode, and the reference electrode is set relative to the others of the working electrode, the counter electrode, and the reference electrode in order to optimize performance of the oxygen sensor assembly.

14. The reference electrodes probe of claim 12, wherein the reference electrode comprises a AgCl coating on a bare Ag wire.

15. The probe of claim 1, wherein the pressure sensor assembly comprises a pressure sensing element and a pressure sensor chamber that is not perfectly round after the cannula has been pre-treated, due to an internal structure near the pressure sensing element, or both.
